(19) United States
(12) Patent Application Publication
(10) Pub. No.: US 2023/0079246 A1
(43) Pub. Date: Mar. 16, 2023

(54) PATIENT MOBILITY CLASSIFICATION

(71) Applicant: Hill-Rom Services, Inc., Batesville, IN (US)

(72) Inventors: Timothy J. RECEVEUR, Apex, NC (US); Sinan BATMAN, Cary, NC (US); Marion LE GALL, Vannes (FR); Eugene URRUTIA, Apex, NC (US)

(21) Appl. No.: 17/895,545

(22) Filed: Aug. 25, 2022

Related U.S. Application Data
(60) Provisional application No. 63/238,817, filed on Aug. 31, 2021.

(51) Int. Cl. A61B 5/11; A61B 5/00

(52) U.S. Cl. CPC A61B 5/1116; A61B 5/1128; A61B 5/6891

(57) ABSTRACT

A patient support apparatus comprises a plurality of load cells, a frame supported on the load cells, a mattress, a plurality of air pressure sensors, and a control system. The mattress includes a plurality of inflatable zones positioned on the frame, the mattress and frame cooperating to direct any patient load through the mattress and frame to the load cells. Each of the plurality of air pressure sensors measures the pressure in a respective inflatable zone of the mattress. The control system includes a controller operable to receive a separate signal from each of the plurality of load cells and each of the plurality of air pressure sensors and process the signals to identify motion of the patient. A motion classifier assesses major and minor motions based on the amplitude and frequency of patient movement and determines a mobility score for the patient.

### PRIORITY CLAIM

This application claims priority under 35 U.S.C. § 119(e) to U.S. Provisional Application No. 63/238,817, filed Aug. 31, 2021, which is expressly incorporated by reference herein.

### BACKGROUND

The present disclosure relates to the use of sensors of a patient support apparatus, such as a hospital bed, for example, to detect patient motion and characterize the patient motion. More specifically, the present disclosure is directed to a motion classifier which assesses major and minor motions based on the amplitude and frequency of patient movement and determines a mobility score for the patient.

The use of load cells in patient support apparatuses, such as hospital beds, for example, to measure patient weight is known. Over time, approaches to using the information from the load cells to detect patient movement and to issue an alert or notification when the patient moves beyond a particular threshold have been developed. The use of load cells to make these determinations and inferences based on the motion or movement is limited by the potential for external influences, such as the addition of equipment to the frame supported on the scale. When this is done, the existing information regarding the position of the patient is compromised as the weight distribution is changed unexpectedly.

The pressure sensors used to measure air pressure in zones of an inflatable mattress are used to control the inflation pressure in the zones to control the interface pressure experienced by a patient supported on the mattress. However, because of transient effects and lack of precision, air pressure sensors associated with mattress zones are not regularly used to measure patient information. The challenges of using air pressure sensors are exacerbated by the variability between the anthropometric characteristics of patients. People with completely different body types sometimes have similar weights. The variations in the surface area of bodies can vary the pressure and volume effects applied to inflatable zones. Still further, the variability in the construction of chambers in the zones makes every application necessary to characterize.

In addition, caregivers or visitors may intermittently apply pressure to the mattress, thereby changing air pressure measurements and the distribution of the weight on the frame. Motion algorithms generally rely on changes in the distribution of weight over multiple sensors to determine patient location and relative movement. These transient and external forces confound the algorithms used to determine patient movement and motion.

In some cases, it is important to determine patient movement relative to the patient support apparatus. Movement in this context means a change in position of the patient on the patient support apparatus, such as rolling over or moving toward an edge of a patient support apparatus to exit the patient support apparatus. A patient's mobility may be indicative of a patient's health and/or medical needs.

Thus, there is a need to improve the approaches to measuring and characterizing a patient's mobility and to determine a patient's mobility score. Improving the identification of instances when there is no motion is important to accurately assess a patient's mobility. A patient's mobility score may depend on the extent of patient movements and the frequency of patient movements.

### SUMMARY

The present disclosure includes one or more of the features recited in the appended claims and/or the following features which, alone or in any combination, may comprise patentable subject matter.

According to a first aspect of the present disclosure, a patient support apparatus comprises a plurality of load cells, a frame supported on the load cells, a mattress including a plurality of inflatable zones positioned on the frame, the mattress and frame cooperating to direct any patient load through the mattress and frame to the load cells, a plurality of air pressure sensors, each air pressure sensor measuring the pressure in a respective inflatable zone of the mattress, and a control system including a controller, the controller operable to receive a separate signal from each of the plurality of load cells and each of the plurality of air pressure sensors to monitor energy detected by each of the load cells and each of the air pressure sensors, and the controller is further operable to process the signals to predict, a mobility score for the patient. The mobility score of the patient is characterized by patient movement amplitude and patient movement frequency.

In one embodiment of the patient support apparatus, the mobility score of the patient is 4 if the patient has no limitations, wherein the mobility score of the patient is 3 if the patient is slightly limited, wherein the mobility score of the patient is 2 if the patient is very limited, and wherein the mobility score of the patient is 1 if the patient is completely limited.

In one embodiment of the patient support apparatus, the patient movement amplitude comprises either slight movement or major movement. In some embodiments of the patient support apparatus, the patient movement frequency comprises number of movements per hour ranging between no movement, occasional movement and frequent movement.

In one embodiment of the patient support apparatus, wherein the mobility score of the patient is determined by using one or more continuous sensors monitoring patient movement on the patient support apparatus. In some embodiments of the patient support apparatus, the patient movement amplitude comprises slight movement when the patient movement does not comprise a postural change for a period of about 30 seconds. In other embodiments of the patient support apparatus, wherein the patient movement amplitude comprises major movement when the patient movement comprises a postural change in a period of about 30 seconds.

In one embodiment of the patient support apparatus, the patient has a mobility score of 4 if the patient has six or more slight movements per hour and one or more major movements per hour, the patient movement amplitude comprises slight movement when the patient movement does not comprise a postural change for a period of about 30 seconds, and the patient movement amplitude comprises major movement when the patient movement comprises a postural change in a period of about 30 seconds.

In one embodiment of the patient support apparatus, the patient has a mobility score of 3 if the patient has six or more slight movements per hour and less than one major movement per hour. The patient movement amplitude comprises slight movement when the patient movement does not comprise a postural change for a period of about 30 seconds, and the patient movement amplitude comprises major movement when the patient movement comprises a postural change in a period of about 30 seconds.

In one embodiment of the patient support apparatus, the patient has a mobility score of 2 if the patient has more than two slight movements per hour and less than one major movement per hour. The patient movement amplitude comprises slight movement when the patient movement does not comprise a postural change for a period of about 30 seconds, and the patient movement amplitude comprises major movement when the patient movement comprises a postural change in a period of about 30 seconds.

In one embodiment of the patient support apparatus, the patient has a mobility score of 1 if the patient has two or less slight movements per hour and less than one major movement per hour. The patient movement amplitude comprises slight movement when the patient movement does not comprise a postural change for a period of about 30 seconds, and the patient movement amplitude comprises major movement when the patient movement comprises a postural change in a period of about 30 seconds.

In one embodiment of the patient support apparatus, the one or more continuous sensor comprises video recording.

According to a first aspect of the present disclosure, a system comprises a patient support surface including a plurality of inflatable zones, a plurality of load cells supporting the patient support surface, a plurality of air pressure sensors, each pressure sensor measuring the pressure in a respective inflatable zone of the patient support surface, and a controller operable to receive a separate signal from each of the plurality of load cells and each of the plurality of air pressure sensors, to process the signals to determine a mobility score of the patient. The mobility score of the patient is characterized by patient movement amplitude and patient movement frequency.

In one embodiment of the system, the controller is operable to determine the mobility score of 4 if the patient has no limitations, the mobility score of 3 if the patient is slightly limited, the mobility score of 2 if the patient is very limited, and the mobility score of 1 if the patient is completely limited. In some embodiments of the system, patient movement amplitude comprises either slight movement or major movement. In other embodiments of the system, the mobility score of the patient is determined by using one or more continuous sensors monitoring patient movement on the patient support apparatus.

In one embodiment of the system, the patient movement frequency comprises number of movements per hour ranging between no movement, occasional movement and frequent movement. In some embodiments of the system, the patient movement amplitude comprises slight movement when the patient movement does not comprise a postural change for a period of about 30 seconds. In other embodiments of the system, the patient movement amplitude comprises major movement when the patient movement comprises a postural change in a period of about 30 seconds.

In one embodiment of the system, the patient has a mobility score of 4 if the patient has six or more slight movements per hour and one or more major movements per hour. The patient movement amplitude comprises slight movement when the patient movement does not comprise a postural change for a period of about 30 seconds, and the patient movement amplitude comprises major movement when the patient movement comprises a postural change in a period of about 30 seconds.

In one embodiment of the system, the patient has a mobility score of 3 if the patient has six or more slight movements per hour and less than one major movement per hour. The patient movement amplitude comprises slight movement when the patient movement does not comprise a postural change for a period of about 30 seconds, and the patient movement amplitude comprises major movement when the patient movement comprises a postural change in a period of about 30 seconds.

In one embodiment of the system, the patient has a mobility score of 2 if the patient has more than two slight movements per hour and less than one major movement per hour. The patient movement amplitude comprises slight movement when the patient movement does not comprise a postural change for a period of about 30 seconds, and the patient movement amplitude comprises major movement when the patient movement comprises a postural change in a period of about 30 seconds.

In one embodiment of the system, the patient has a mobility score of 1 if the patient has two or less slight movements per hour and less than one major movement per hour. The patient movement amplitude comprises slight movement when the patient movement does not comprise a postural change for a period of about 30 seconds, and the patient movement amplitude comprises major movement when the patient movement comprises a postural change in a period of about 30 seconds.

In one embodiment of the system, the one or more continuous sensor comprises video recording.

According to a third aspect of the present disclosure, a method of determining a mobility score of a patient on a patient support apparatus comprising an inflatable mattress having multiple inflatable zones, the method comprising the steps of monitoring signals from a plurality of pressure sensors, each pressure sensor providing a signal indicative of the pressure in a respective inflatable zone, monitoring signals from a plurality of load cells, the plurality of load cells supporting inflatable mattress, monitoring the energy detected by each of the load cells and each of the pressure sensors, processing the signals from the load cells and pressure sensors to identify any patient movement, and upon detection of patient movement, determining the mobility score based on patient movement amplitude and patient movement frequency.

In one embodiment of the method, the mobility score of the patient is determined to be 4 if the patient has no limitations, wherein the mobility score of the patient is determined to be 3 if the patient is slightly limited, wherein the mobility score of the patient is determined to be 2 if the patient is very limited, and wherein the mobility score of the patient is determined to be 1 if the patient is completely limited.

In one embodiment of the method, the patient movement amplitude comprises either slight movement or major movement. In some embodiments of the method, the patient movement frequency comprises number of movements per hour ranging between no movement, occasional movement and frequent movement. In other embodiments of the method, determining the mobility score of the patient comprises using one or more continuous sensors monitoring patient movement on the patient support apparatus.

In one embodiment of the method, the patient movement amplitude comprises slight movement when the patient movement does not comprise a postural change for a period of about 30 seconds. In some embodiments of the method, the patient movement amplitude comprises major movement when the patient movement comprises a postural change in a period of about 30 seconds.

In one embodiment of the method, the patient has a mobility score of 4 if the patient has six or more slight movements per hour and one or more major movements per hour. The patient movement amplitude comprises slight movement when the patient movement does not comprise a postural change for a period of about 30 seconds, and the patient movement amplitude comprises major movement when the patient movement comprises a postural change in a period of about 30 seconds.

In one embodiment of the method, the patient has a mobility score of 3 if the patient has six or more slight movements per hour and less than one major movement per hour. The patient movement amplitude comprises slight movement when the patient movement does not comprise a postural change for a period of about 30 seconds, and the patient movement amplitude comprises major movement when the patient movement comprises a postural change in a period of about 30 seconds

In one embodiment of the method, the patient has a mobility score of 2 if the patient has more than two slight movements per hour and less than one major movement per hour. The patient movement amplitude comprises slight movement when the patient movement does not comprise a postural change for a period of about 30 seconds, and the patient movement amplitude comprises major movement when the patient movement comprises a postural change in a period of about 30 seconds.

In one embodiment of the method, the patient has a mobility score of 1 if the patient has two or less slight movements per hour and less than one major movement per hour. The patient movement amplitude comprises slight movement when the patient movement does not comprise a postural change for a period of about 30 seconds, and the patient movement amplitude comprises major movement when the patient movement comprises a postural change in a period of about 30 seconds.

In one embodiment of the method, the one or more continuous sensor comprises video recording.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a perspective view of a patient support apparatus including a control system operable to measure signals from a plurality of sensors and process those signals according to the present disclosure;

FIG. 2 is a block diagram of a portion of the control system of the patient support apparatus of FIG. 1;

FIG. 3 is a diagrammatic illustration of the interaction between a first frame of the patient support apparatus of FIG. 1 and a second frame supported on load cells supported from the first frame;

FIG. 4, is a side view of a portion of the patient support apparatus of FIG. 1 showing a first frame supported on load cells supported on a second frame, the load cells supporting all of the load of the first frame;

FIG. 5 is illustrates a generalized algorithm for processing sensor data and determining a mobility score from one or more sensors on the patient support apparatus;

FIG. 6 illustrates a rule-based algorithm implemented to determine a patient mobility score of a patient on the patient support apparatus.

### DETAILED DESCRIPTION

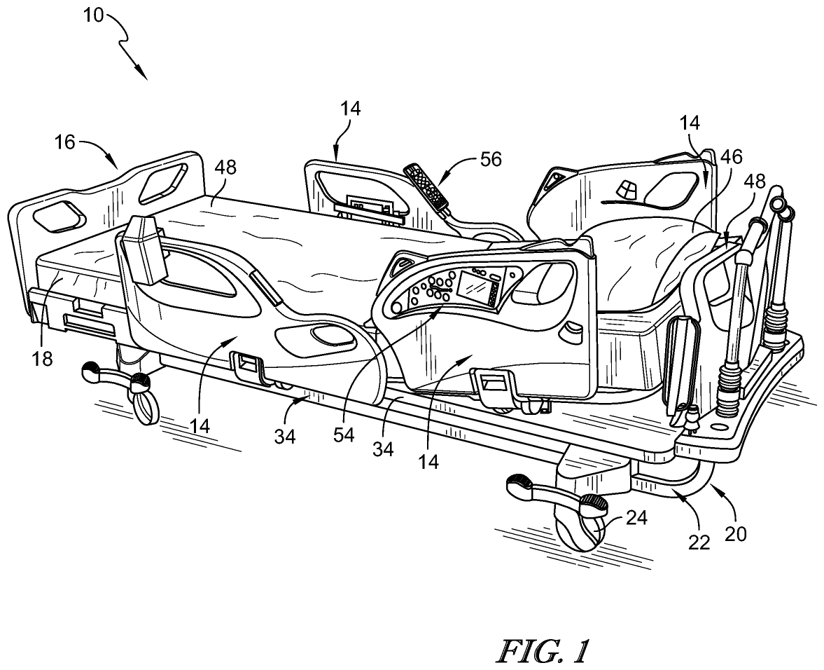

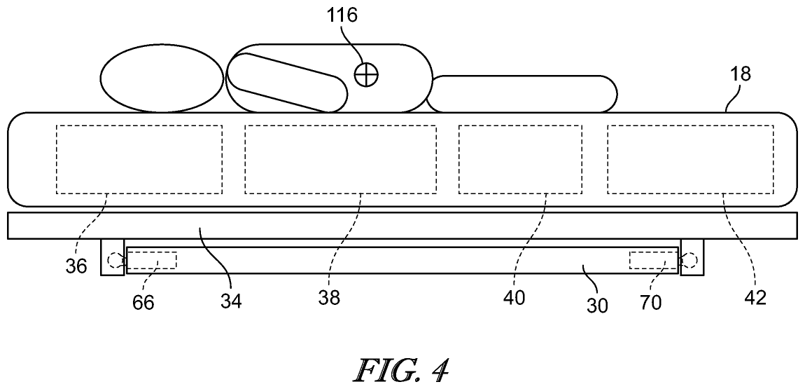

An illustrative patient support apparatus 10 embodied as a hospital bed is shown in FIG. 1. The bed 10 of FIG. 1 has a frame 20 which includes a base frame 22 supported on casters 24. The stationary base frame 22 further supports a weigh frame 30 that an adjustably positionable mattress support upper frame 34 supporting a mattress 18. As shown in FIG. 4, the illustrative mattress 18 is an inflatable patient support surface which includes inflatable zones including a head zone 36, a seat zone 38, thigh zone 40, and a foot zone 42. The bed 10 further includes a headboard 12 at a head end 46 of the bed 10, a footboard 16 at a foot end 48 of the bed 10, and a movable siderails 14 coupled to the upper frame 34 of the bed 10. The bed 10 also includes a user interface 54 positioned on one of the siderails 14. The bed 10 of the embodiment of FIG. 1 is conventionally configured to adjustably position the upper frame 34 relative to the base frame 22 to adjust the position of a patient supported on the mattress 18.

Conventional structures and devices may be provided to adjustably position the upper frame 34, and such conventional structures and devices may include, for example, linkages, drives, and other movement members and devices coupled between base frame 22 and the weigh frame 30, and/or between weigh frame 30 and upper frame 34. Control of the position of the upper frame 34 and mattress 18 relative to the base frame 22 or weigh frame 30 is controlled, for example, by a patient control pendant 56 or user interface 54. The upper frame 34 may, for example, be adjustably positioned in a general incline from the head end 46 to the foot end 48 or vice versa. Additionally, the upper frame 34 may be adjustably positioned such that the head section 44 of the mattress 18 is positioned between minimum and maximum incline angles, e.g., 0-65 degrees, relative to horizontal or bed flat, and the upper frame 34 may also be adjustably positioned such that a seat section (not shown) of the mattress 18 is positioned between minimum and maximum bend angles, e.g., 0-35 degrees, relative to horizontal or bed flat. Those skilled in the art will recognize that the upper frame 34 or portions thereof may be adjustably positioned in other orientations, and such other orientations are contemplated by this disclosure.

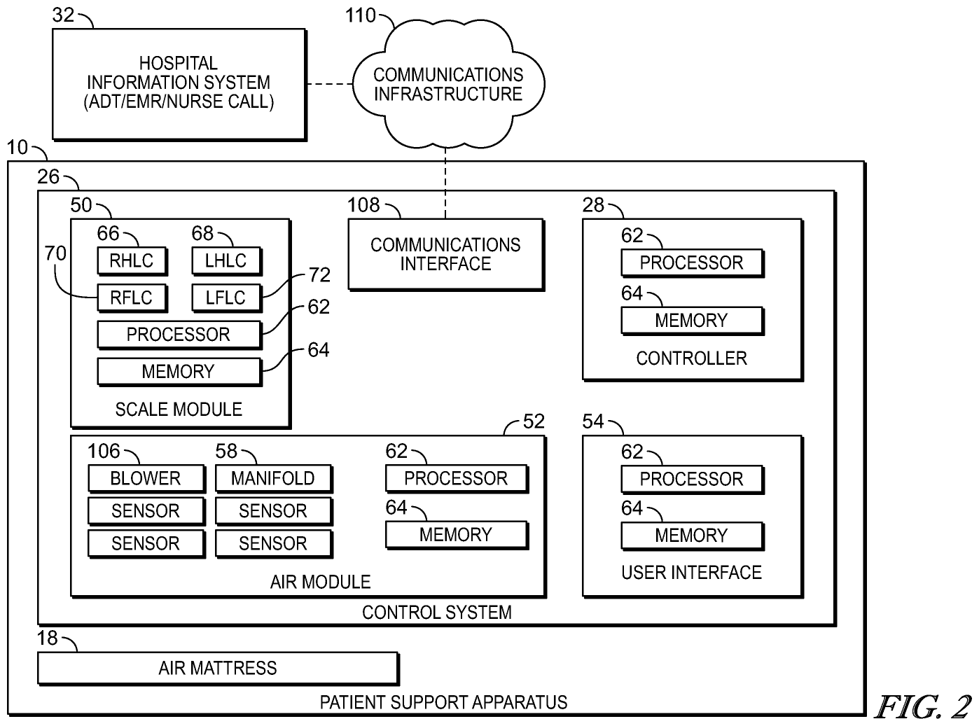

In one illustrative embodiment shown diagrammatically in FIG. 2, the bed 10 has a control system 26 that includes a controller 28, a scale module 50, an air module 52, and the user interface 54. In the illustrative embodiment each of the controller 28, scale module 50, air module 52, and user interface 54 includes a processor 62 and a memory device 64. The processor 62 and memory device 64 are shown only with respect to the controller 28, but similar structures are used in the scale module 50, air module 52, and user interface 54. The memory device 64 includes instructions that, when executed by the processor 62, cause the processor 62 to perform functions as associated with the particular one of controller 28, scale module 50, air module 52, and user interface 54. The components of the control system 26 communicate amongst themselves to share information and distribute the functions of the bed 10. The processor 62 of each of the controller 28, scale module 50, air module 52, and user interface 54 is also operable, based on instructions from the memory device 64, to communicate with the others of the controller 28, scale module 50, air module 52, and user interface 54 using a communications protocol. It should be understood that the term processor here includes any microprocessor, microcontroller, processor circuitry, control circuitry, preprogrammed device, or any structure capable of accessing the memory device and executing non-transient instructions to perform the tasks, algorithm, and processed disclosed herein. In the illustrative embodiment, the control system 26 employs a conventional controller area network (CAN) for communications between subsystems, but it should be understood that any of a number of networking and communications solutions could be employed in the control system 26.

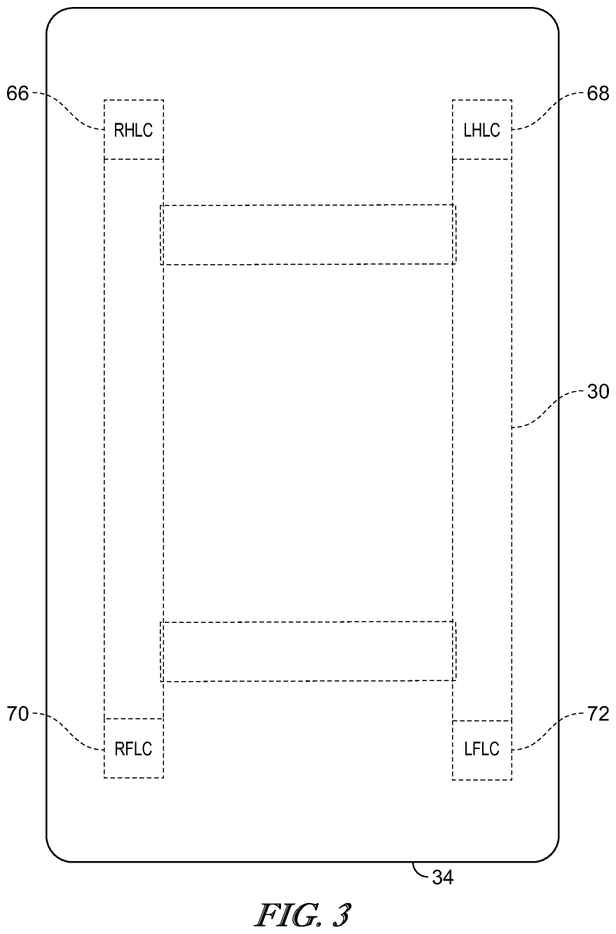

As shown in FIG. 3, the scale module 50 includes four load cells 66, 68, 70, and 72. To determine a weight of a patient supported on the mattress 18, the load cells 66, 68, 70, and 72 are positioned between the weigh frame 30 and the upper frame 34 as illustrated in FIGS. 3 and 4. Each load cell 66, 68, 70, 72 is configured to produce a signal indicative of a load supported by the respective load cell 66, 68, 70, 72 from the upper frame 34 relative to the weigh frame 30. Some of the structural components of the bed 10 will be designated hereinafter as “right”, “left”, “head” and “foot” from the reference point of an individual lying on the individual's back on the mattress 18 with the individual's head oriented toward the head end 46 of the bed 10 and the individual's feet oriented toward the foot end 48 of the bed 10. Following this convention, the load cell 66 is designated as the right head load cell (RHLC) in the figures to represent that the load cell 66 is positioned at the right side of the bed 10 at the head end 46. The load cell 68 is designated at the left head load cell (LHLC), the load cell 70 is designated as the right foot load cell (RFLC), and the load cell 72 is designated left foot load cell (LFLC), each following the same convention.

The scale module 50 includes a processor 62 that is in communication with each of the respective load cells 66, 68, 70, and 72 and operable to process the signals from the load cells 66, 68, 70, and 72. The memory device 64 is also utilized by the controller 28 to store information corresponding to features and functions provided by the bed 10.

A weight distribution of a load among the plurality of load cells 66, 68, 70, and 72 may not be the same depending on variations in the structure of the bed 10, variations in each of load cells 66, 68, 70, and 72 and the position of the load on the mattress 18 relative to the particular load cell 66, 68, 70, or 72. Accordingly, a calibration constant for each of the load cells 66, 68, 70, and 72 is established to adjust for differences in the load cells 66, 68, 70, and 72 in response to the load borne by each. Each of the load cells 66, 68, 70, and 72 produces a signal indicative of the load supported by that load cell 66, 68, 70, or 72. The loads detected by each of the respective load cells 66, 68, 70, 72 are adjusted using a corresponding calibration constant for the respective load cell 66, 68, 70, 72. The adjusted loads are then combined to establish the actual weight supported on the bed 10. In the present disclosure, the independent signals from each of the load cells 66, 68, 70, 72 is used to draw inferences about the movement and motion of the patient.

The air module 52 is the functional controller for the mattress 18 and includes processor 62 and a memory device 64. The processor 62 is in communication with a blower 106, a manifold 58, and an air pressure sensor assembly 60. The air module 52 is a conventional structure with the manifold 58 operating under the control of the processor 62 to control the flow of air from the blower 106 into and out of the head zone 36, seat zone 38, thigh zone 40, and foot zone 42 to control the interface pressure experienced by the patient supported on the mattress 18. The sensor assembly 60 includes separate sensors for measuring the air pressure in each of the head zone 36, seat zone 38, thigh zone 40, and foot zone 42. The pressure sensor assembly includes a head zone sensor 82, a seat zone sensor 84, a thigh zone senor 86, and a foot zone sensor 88. While signals from the sensors 82, 84, 86, and 88 are used to control the pressure in the respective zones, applying the principles of the present disclosure, the signals are also useful in making inferences regarding patient movement and, when used synergistically with the information gleaned from the signals from the load cells 66, 68, 70, and 72, provide a more fulsome and accurate analysis of patient movement and/or any motion associated with the patient support apparatus.

The scale module 50 and air module 52 of the bed 10 are used for measuring the motions of a patient that occupies the bed 10. Referring to FIG. 4, a diagrammatic side view of a patient supported on the mattress 18 and frame 34 with the load of the patient being borne by the inflated zones 36, 38, 40, and 42 and passed through the mattress to the frame 34 to the load cells 66, 68, 70, and 72 supported from the weigh frame 30. As seen in FIG. 4, in a static condition, the patient's weight is appropriately distributed over the inflated zones 36, 38, 40, and 42. Each of those zones 36, 38, 40, and 42 are inflated to a target pressure based on the patient's weight detected by the scale module 50 and the expected distribution of the patient.

Through an empirical study that included real-time data collection from video observation of test subject patients synchronized with signals from load cells of the scale module of the bed supporting the test subject patients, the types of motion from the where classified in one of three types: lateral patient motions (LPMs); vertical or self-offloading patient movements (SOs); or non-patient motion artifacts (NPMAs). There were also observations that found that load cell signals varied when there was no patient movement. These artifacts were designated as non-movements (NMs). Permutations of these categories, called “complex movements”, also including further categorization into combinations including different directionality of the simple movements was also established.

The patient and the bed 10 are treated as a closed system. The total energy of the closed system will be constant and conserved over time of typical patient movements. Any energy that is created by the patient as a result of them moving does not change the overall loading of all four load cells 66, 68, 70, and 72, but simply changes the proportion the total load that each load cell 66, 68, 70, or 72 is carrying at any given time. There is no total gain of loads, the loads simply shift around the four load cells 66, 68, 70, and 72 as the patient moves laterally.

In contrast, when a caregiver pushes or pulls on the patient or bed 10 (a NPMA), the closed-system is corrupted by an external energy source and the net load on the load cells 66, 68, 70, and 72 increased or decreased. This is the case for both transient touches of the bed 10, such as when a person hugs the patient, and in sustained touches of the bed 10, such as when a caregiver leans on bed 10 while doing long procedure. In either case, an additional load is introduced to the load cells 66, 68, 70, and 72 resulting in a material change from the sum of the loads on each load cell 66, 68, 70, and 72 when the transient load is applied to the bed 10. The value of the transient load, designated as total transient load (TTL) is calculated by subtracting from the total load measured by the load cells 66, 68, 70, and 72, the closed-system load measured before the transient event; the closed-system load which is effectively the patient's static weight, designated as the DC sum of beams (DC SB) which may be determined using known techniques, such as that disclosed in U.S. Pat. No. 10,054,479 titled “BED WITH AUTOMATIC WEIGHT OFFSET DETECTION AND MODIFICATION”, which is incorporated herein for the disclosure of monitoring and updating a patient load to establish a static patient weight, DC SB.

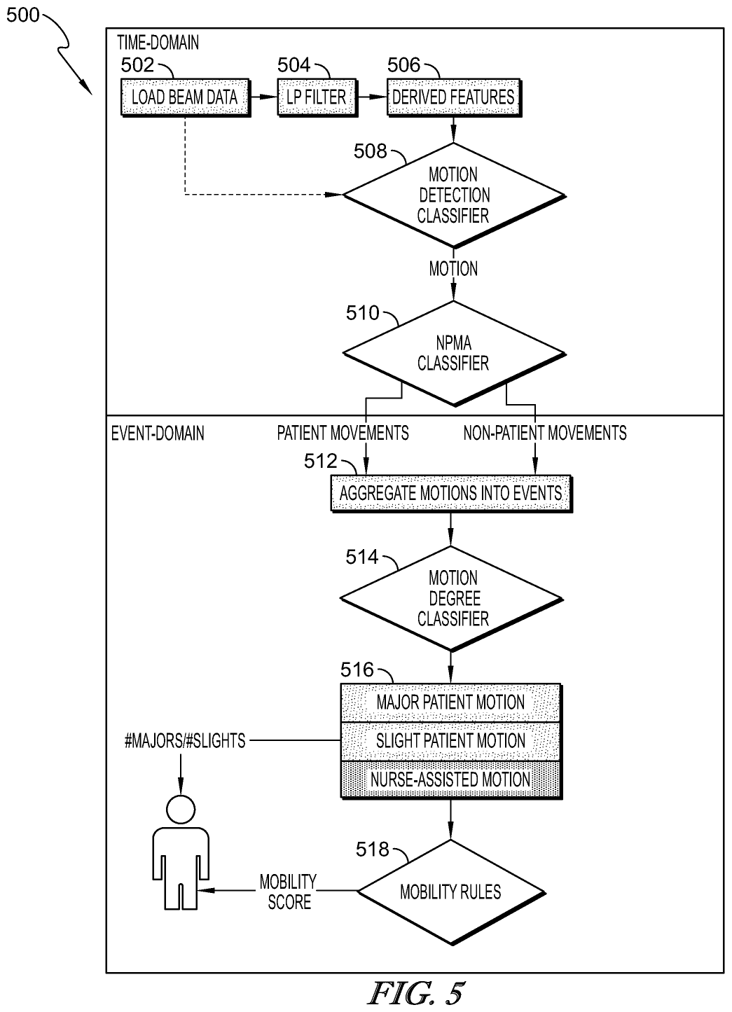

Once the motion is identified as a patient movement or a non-patient movement, the patient movements are aggregated into events and the degree of motion is classified. The degree of motion may be major patient motion, slight patient motion, and/or nurse-assisted motion. The present disclosure is directed to methods and systems for a motion classifier that assesses patient movement to determine the degree of motion and then uses the identified degree of motion and motion frequency to determine a patient mobility score. A generalized algorithm 500 for processing sensor data and determining a mobility score from one or more sensors on a bed 10 is developed as shown in FIG. 5.

Sensor data including load beam data from a bed 10 is assessed at step 502 and passed through a low pass filter at step 504 and distinguishing features based on one or more variables are identified at step 506. Once the DCSB or patient weight is established, a simple threshold can be tested to determine whether a TTL is a NPMA or not. Patient motion is predicted by one variable at step 508, and the difference between NPMA/non-NPMA is determined by at least two variables at step 510. An approach serializes classifiers by determining if there is any motion (patient or non-patient) and passes that data into a classifier that determines the presence of NPMA vs. non NPMA at step 510. Steps 502-510 may be implemented in the time domain.

Once the various patient movements are classified as NPMA or non-NPMA, the patient movements are aggregated at step 512 and analyzed at step 514 in the event domain. The degree of motion is determined at step 514. If NPMA is identified, then the signal data is disregarded. The information is then moved to a database associated with the patient as step 516. For example, at step 516 the patient's medical record can be updated, based on the inference identify, objectively, the patient's motion and behavior such as major patient motion, slight patient motion, or ingress or egress with or without caregiver assistance. Mobility rules are applied at step 518 on the set of data obtained at step 516 and used to determine a patient mobility score at step 518.

In one embodiment, the determination of major patient movement vs slight patient movement vs nurse assisted patient movement motion at step 516 is based on the nurse assessment of the Braden score and/or Braden mobility sub-score of the aggregated motions. The mobility subscale of the Braden scale defines the four mobility levels. Completely immobile is defined as when the subject does not make even slight changes in body or extremity position without assistance. Very limited movement is defined as when the subject makes occasional slight changes in body or extremity position but unable to make frequent or significant changes independently. Slightly limited movement is defined as when the subject makes frequent though slight changes in body or extremity position independently. No limitation is defined as when the subject makes major and frequent changes in position without assistance. Thus, mobility may be characterized by movement amplitude (slight or major movement) and movement frequency (number of movements per hour ranging between no movement, occasional movement and frequent movement).

To get an initial set of qualitative data, a set of data was created, using healthy participants performing a list of 22 movements. The 22 movements were reproduced in a laboratory setting and then scored individually in terms of amplitude (categorized by no movement, slight movement and major movement). A Kinect recording was used to document limb and body displacement in order to characterize each movement.

The recording (10-15 sec) was played and then the nurses were asked to score the movements. Each movement amplitude was scored separately by 3 nurses of ICU and/or Medical-Surgical background based on the scoring system. The score options were 0 if the nurse considered the test subject on the video was not doing a movement, 1 if the nurse considered the test subject on the video was doing a slight movement, and 2 if the nurse considered the test subject on the video was doing a major movement. It was ensured that the score fit an ICU or Medical-Surgical patient. The scoring was based on nurse interviews and on multiple parameters, as detailed by literature (EPUAP, NPIAP, PPPIA Clinical Guidelines 2019, chap. Risk factors and risk assessment). There was some variability in the scoring of the aggregated motions between the nurses. A consensus meeting with 4 nurses was scheduled to discuss the movements and an amplitude scoring system was determined. The question, “Is this movement helping to prevent injury?” was answered with every movement. The final results are documented Table 1. This set of data was important to reproducibly test different body morphotypes and comprehend the output of the sensors of bed 10.

TABLE 1

| | | Amplitude | | |
|---|---|---|---|---|
| | Movement | movement | | |
| No | description | score | Comments | No |
| 1 | Turn head without | 1 | Really close to be a no | 1 |
| | raise head | | movement if looking | |
| | | | for skin perspective | |
| 2 | Raise head and | 1 | Rigidity of the patient | 2 |
| | turn head | | body something to | |
| | | | notice | |
| 3 | Left hand below | 1 | NA | 3 |
| | the head | | | |
| 4 | Cross arms over | 1 | Both arms could mean | 4 |
| | the stomach | | more than slight | |
| | | | movement, | |
| | | | neurological significant | |
| 5 | Leg to the edge of | 1 | NA | 5 |
| | the bed | | | |
| 6 | Cross the legs | 1 | NA | 6 |
| 7 | Bend the left knee | 1 | NA | 7 |
| | on the bed | | | |
| 8 | Bend the left knee | 1 | Maybe a 2 under a | 8 |
| | on the heel | | specific under context | |
| | | | if patient arms are tied | |
| | | | (because overcoming | |
| | | | gravity), neurological | |
| | | | good mvt | |
| 9 | Lifting of pelvis | 1 | Consider major | 9 |
| | without moving legs | (MS)- | movement because of | |
| | | 2 | offload of the pelvis for | |
| | | (ICU) | ICU nurse - no | |
| | | | consensus achieved | |
| 10 | Pelvis to the left | 1 | NA | 10 |
| | then to the right | | | |
| 11 | Shift of trunk | 1 | May not be purposeful, | 11 |
| | without moving | | movement when patient | |
| | legs | | cough - Abnormal | |
| | | | position in the bed | |
| 12 | Raise head and | 1 | NA | 12 |
| | shoulders to the | | | |
| | left then to the | | | |
| | right | | | |
| 13 | Weight on the left | 1 | Abnormal posture, ET | No |
| | shoulder without | | tube? | |
| | moving legs | | | |
| 14 | Weight on | 2 | Offloading but come | No (because patient |
| | shoulders and | | back to his initial | rolls backwards), |
| | bend the legs | | position | need more info. |
| 15 | Lift upper body | 2 | NA | Maybe, Not |
| 16 | then | | | sufficient but by |
| | Tender arm to | | | analyzing this mvt, |
| | catch something | | | the patient should be |
| | | | | able to move and |
| | | | | offload himself |
| | | | | (patient came back |
| | | | | in the same |
| | | | | position), not |
| | | | | assistance needed |
| | | | | but may need |
| | | | | encouragement |
| 17 | Turn slightly on | 2 | NA | Yes |
| | the left flank | | | |
| 18 | Raise the bust and | 2 | NA | Yes |
| | turn on the left | | | |
| | flank | | | |
| 19 | Raise the bust and | 2 | Ready for discharge, | Yes |
| | turn on the | | rolling to their stomach | |
| | stomach | | and center himself, | |
| | | | coordinate, smooth and | |
| | | | strength, prone, balance | |
| | | | for extremities | |
| 20 | Sit without | 2 | Core strength | Yes (careful, may be |
| | moving legs | | | an agitation |
| | | | | movement) |
| 21 | Sit and cross legs | 2 | NA | Yes |
| 22 | Sit on the edge of | 2 | NA | Yes |
| | the bed | | | |

*NA—no nurse comment

In some embodiments, one or more continuous sensors may be used to monitor patient movement. The one or more continuous sensors used to monitor patient movement may capture more information than a nurse or a caregiver coming several times in to a patient room. In some embodiments, movement monitored by the one or more continuous sensors may be used and processed by the controller 28. There is minimal literature qualitatively documenting patient bed activity. However, patient sleep activity has been reported to include both, the patient movements per night and the frequency of such patient movements. Patient bed activity may be at its lowest during sleep as the body is offloading the stress of the day. Thus, in some embodiments, a threshold based on patient sleep activity used to correspond to the extreme minimal value of patient movement may be appropriate for a patient who is in pain or is dazed with medication.

As shown in Table 2, Verhaert et al. (2011) documented healthy subject sleep activity for 4 nights. The 15 subjects (9 males, 6 females) were aged between 25.8±8.6 years, with an average body mass index of 22.0±2.5 kg/m2 (range: 18.4 and 29.4 kg/m2). The amount of body movements was determined to be the amount of movements in bed without posture change. The postural immobility (PI) was defined as episodes of immobility punctuated by major postural shifts and showed the organization of these periods to be periodic and related to the sleep cycle phase. Quantitatively, the periods of immobility (PI) were defined as episodes of 30 min or more without any occurrence of movement. Analogously, periods of postural immobility were periods of 30 min or more without any posture changes.

TABLE 2

| | Mean | Standard Deviation |
|---|---|---|
| Body movement per minute | 0.162 | 0.071 |
| Postural change per minute | 0.034 | 0.019 |
| Maximal PI (min) | 48.0 | 14.8 |
| Maximal PPI (min) | 108.5 | 46.4 |
| Time in bed (min) | 465.0 | 22.9 |
| Average PI per night | 7.0 | 2.8 |
| Average PPI per night | 37.1 | 25.8 |

Based on the study done by Verhaert et al. and the assessment done by the nurses described above, it was extrapolated that a postural change will be required for any movement to be classified as a major movement, i.e. a postural change will be classified as a major movement. Furthermore, any body movement recorded by the Verhaert's team will be equivalent to a slight movement. Thus, from Table 2, it was determined that about 10±4 slight movements per hour, and about 2±1 major movements per hour were detected for a healthy, sleeping subject.

Based on the definition of each sub-score of the mobility, a patient with no limitation (a score 4) will be determined if the patient has 6 or more slight movements per hour and have one or more major movements per hour. A slightly limited patient (a score 3) will be determined if the patient makes frequent though slight changes in body or extremity position independently. Thus, the patient may have 6 or more slight movements per hour and less than one major movements per hour. A very limited patient (a score 2) will be determined if the patient makes occasional slight changes in body or extremity position but is unable to make frequent or significant changes independently.

In some embodiments, the number of occasional slight changes in a patient's body or extremity position may be difficult to interpret. However, if the data of the patient does not fit into score 3 or score 4, the patient may be determined to be either very limited or completely immobile. A completely immobile patient (a score 2) will be determined if the patient is not making even slight changes in body or extremity position without assistance. Thus, the patient may have less than or equal to 2 slight movements per hour and less than one major movement per hour. 2 was chosen arbitrarily to support the fact that the patient may move his/her extremities a little without the caregiver notice. In some embodiments, a different criteria may be used to determine a completely immobile patient.

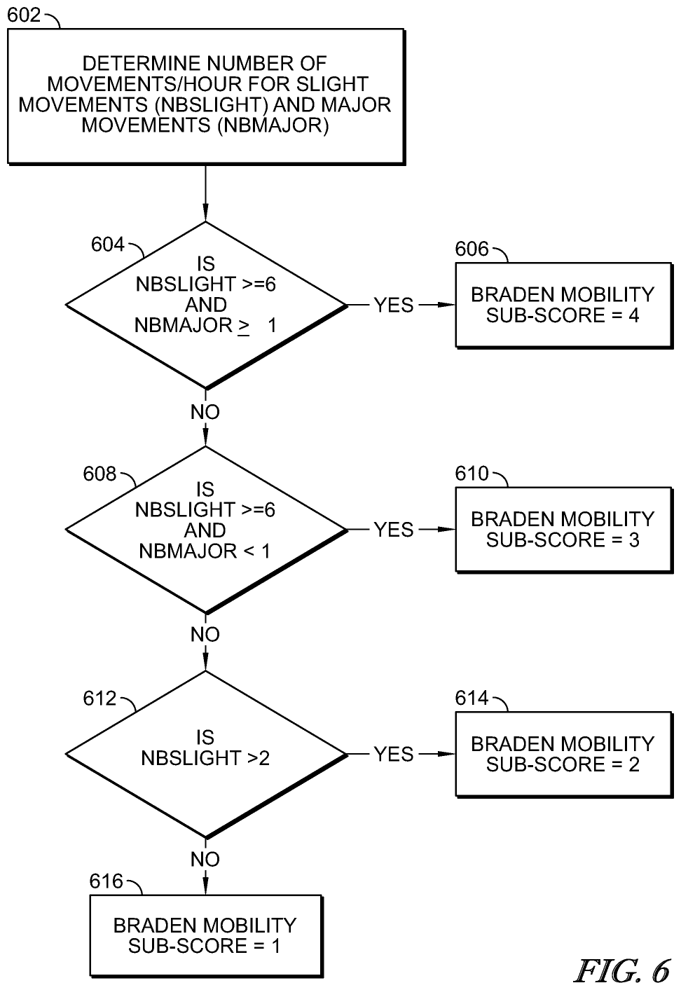

In one embodiment, a rule-based algorithm illustrated in FIG. 6 may be implemented to determine a patient mobility score of a patient on bed 10. Steps 606, 610, 614, 616 are the Braden mobility sub-scores. Steps 604, 608, 612 corresponds to the following rules used to determine the Braden mobility sub scores. The number of movements per hour of slight movements and major movements is assessed in step 602. In step 604, if the number of slight movements is determined to be greater than or equal to 6 and if the number of major movements is determined to be greater than or equal to 1, step 606 is executed. In step 606, the Braden mobility sub-score is determined to be 4. If the number of slight movements is determined to be less than 6 or if the number of major movements is determined to be less than 1 in step 604, step 608 is executed. In step 608, if the number of slight movement is determined to be greater than or equal to 6 and if the number of major movements is determined to be less than 1, step 610 is executed. In step 610, the Braden mobility sub-score is determined to be 3. If the number of slight movements is determined to be less than 6 in step 608, step 612 is executed. In step 612, if the number of slight movement is determined to be greater than 2, step 614 is executed. In step 614, the Braden mobility sub-score is determined to be 2. If the number of slight movements is determined to be less than or equal to 2 in step 612, step 616 is executed. In step 616, the Braden mobility sub-score is determined to be 1.

This rule-based algorithm may be used to assess the Braden mobility sub-score, based on detection of patient movement (without including NPMA) and classification of the patient movement as slight or major movement. In some embodiments, this rule-based algorithm may be used to assess the Braden mobility sub-score, without including any NPMA while detecting patient movement.

Although this disclosure refers to specific embodiments, it will be understood by those skilled in the art that various changes in form and detail may be made without departing from the subject matter set forth in the accompanying claims.

1. A patient support apparatus located on a floor comprising:
a plurality of load cells,
a frame supported on the load cells,
a mattress including a plurality of inflatable zones positioned on the frame, the mattress and frame cooperating to direct any patient load through the mattress and frame to the load cells,
a plurality of air pressure sensors, each air pressure sensor measuring the pressure in a respective inflatable zone of the mattress, and
a control system including a controller, the controller operable to receive a separate signal from each of the plurality of load cells and each of the plurality of air pressure sensors to monitor energy detected by each of the load cells and each of the air pressure sensors, and the controller is further operable to process the signals to predict, a mobility score for the patient, and
wherein the mobility score of the patient is characterized by patient movement amplitude and patient movement frequency.

2. The patient support apparatus of claim 1, wherein the mobility score of the patient is 4 if the patient has no limitations, wherein the mobility score of the patient is 3 if the patient is slightly limited, wherein the mobility score of the patient is 2 if the patient is very limited, and wherein the mobility score of the patient is 1 if the patient is completely limited.

3. The patient support apparatus of claim 1, wherein the patient movement amplitude comprises either slight movement or major movement.

4. The patient support apparatus of claim 1, wherein the patient movement frequency comprises number of movements per hour ranging between no movement, occasional movement and frequent movement.

5. The patient support apparatus of claim 1, wherein the mobility score of the patient is determined by using one or more continuous sensors monitoring patient movement on the patient support apparatus.

6. The patient support apparatus of claim 3, wherein the patient movement amplitude comprises slight movement when the patient movement does not comprise a postural change for a period of about 30 seconds.

7. The patient support apparatus of claim 3, wherein the patient movement amplitude comprises major movement when the patient movement comprises a postural change in a period of about 30 seconds.

8. The patient support apparatus of claim 1, wherein the patient has a mobility score of 4 if the patient has six or more slight movements per hour and one or more major movements per hour,
wherein the patient movement amplitude comprises slight movement when the patient movement does not comprise a postural change for a period of about 30 seconds, and
wherein the patient movement amplitude comprises major movement when the patient movement comprises a postural change in a period of about 30 seconds.

9. The patient support apparatus of claim 1, wherein the patient has a mobility score of 3 if the patient has six or more slight movements per hour and less than one major movement per hour,
wherein the patient movement amplitude comprises slight movement when the patient movement does not comprise a postural change for a period of about 30 seconds, and
wherein the patient movement amplitude comprises major movement when the patient movement comprises a postural change in a period of about 30 seconds.

10. The patient support apparatus of claim 1, wherein the patient has a mobility score of 2 if the patient has more than two slight movements per hour and less than one major movement per hour,
wherein the patient movement amplitude comprises slight movement when the patient movement does not comprise a postural change for a period of about 30 seconds, and
wherein the patient movement amplitude comprises major movement when the patient movement comprises a postural change in a period of about 30 seconds.

11. The patient support apparatus of claim 1, wherein the patient has a mobility score of 1 if the patient has two or less slight movements per hour and less than one major movement per hour,
wherein the patient movement amplitude comprises slight movement when the patient movement does not comprise a postural change for a period of about 30 seconds, and
wherein the patient movement amplitude comprises major movement when the patient movement comprises a postural change in a period of about 30 seconds.

12. A system comprising:
a patient support surface including a plurality of inflatable zones,
a plurality of load cells supporting the patient support surface,
a plurality of air pressure sensors, each pressure sensor measuring the pressure in a respective inflatable zone of the patient support surface, and
a controller operable to receive a separate signal from each of the plurality of load cells and each of the plurality of air pressure sensors, to process the signals to determine a mobility score of the patient, and
wherein the mobility score of the patient is characterized by patient movement amplitude and patient movement frequency.

13. The system of claim 12, wherein the mobility score of the patient is determined by using one or more continuous sensors monitoring patient movement on the patient support apparatus.

14. The system of claim 13, wherein the one or more continuous sensor comprises video recording.

15. A method of determining a mobility score of a patient on a patient support apparatus comprising an inflatable mattress having multiple inflatable zones, the method comprising the steps of:
monitoring signals from a plurality of pressure sensors, each pressure sensor providing a signal indicative of the pressure in a respective inflatable zone;
monitoring signals from a plurality of load cells, the plurality of load cells supporting inflatable mattress;
monitoring the energy detected by each of the load cells and each of the pressure sensors;
processing the signals from the load cells and pressure sensors to identify any patient movement;
upon detection of patient movement, determining the mobility score based on patient movement amplitude and patient movement frequency.

16. The method of claim 15, wherein the mobility score of the patient is determined to be 4 if the patient has no limitations, wherein the mobility score of the patient is determined to be 3 if the patient is slightly limited, wherein the mobility score of the patient is determined to be 2 if the patient is very limited, and wherein the mobility score of the patient is determined to be 1 if the patient is completely limited.

17. The method of claim 15, wherein the patient movement amplitude comprises either slight movement or major movement.

18. The method of claim 15, wherein the patient movement frequency comprises number of movements per hour ranging between no movement, occasional movement and frequent movement.

19. The method of claim 15, wherein determining the mobility score of the patient comprises using one or more continuous sensors monitoring patient movement on the patient support apparatus.

20. The method of claim 19, wherein the one or more continuous sensor comprises video recording.
